(19) United States
(12) Patent Application Publication
(10) Pub. No.: US 2021/0363216 A1
(43) Pub. Date: Nov. 25, 2021

(54) BIFUNCTIONAL BINDING POLYPEPTIDES

(71) Applicant: IMMUNOCORE LIMITED, Abingdon, Oxfordshire (GB)

(72) Inventors: Giovanna BOSSI, Abingdon (GB); Carlos REIS, Abingdon (GB); Rajeevkumar TAWAR, Abingdon (GB); Adam CURNOCK, Abingdon (GB); Nicola SMITH, Abingdon (GB)

(21) Appl. No.: 17/055,529

(22) PCT Filed: May 14, 2019
(86) PCT No.: PCT/EP2019/062384

§ 371 Date: Nov. 13, 2020

(30) Foreign Application Priority Data
May 14, 2018 (GB) 1807767.7
Nov. 30, 2018 (GB) 1819584.2

(51) Int. Cl. C07K 14/725; C07K 16/28; C07K 14/705

(52) U.S. Cl. CPC C07K 14/7051; C07K 16/2833; C07K 16/2818; C07K 2317/75; C07K 2317/31; C07K 2317/622; C07K 2319/00; C07K 14/70532

(57) ABSTRACT

The present invention provides bifunctional binding polypeptide comprising a pMHC binding moiety and a PD-1 agonist.

### BACKGROUND

The PD-1 pathway is known to play a vital role in regulating the balance between inhibitory and stimulatory signals in the immune system. Activation of the PD-1 pathway down-regulates immune activity, promoting peripheral immune tolerance and preventing autoimmunity (Keir et al., Annu Rev Immunol, 26:677-704, 2008; Okazaki et al., Int Immunol 19:813-824, 2007). PD-1 is a transmembrane receptor protein expressed on the surface of activated immune cells, including T cells, B cells, NK cells and monocytes (Agata et al., Int Immunol 8:765-772, 1996). The cytoplasmic tail of PD-1 comprises an immunoreceptor tyrosine-based inhibitory motif (ITIM). PD-L1 and PD-L2 are the natural ligands of PD-1 and are expressed on the surface of antigen presenting cells (Dong et al., Nat Med., 5:1365-1369, 1999; Freeman et al., J Exp Med 192:1027-1034, 2000; Latchman et al., Nat Immunol 2:261-268, 2001). Upon ligand engagement, phosphatases are recruited to the ITIM region of PD-1 leading to inhibition of TCR-mediated signaling, and subsequent reduction in lymphocyte proliferation, cytokine secretion and cytotoxic activity. PD-1 may also induce apoptosis in T cells via its ability to inhibit survival signals from co-stimulation (Keir et al., Annu Rev Immunol, 26:677-704, 2008).

The central role of the PD-1 pathway in controlling autoimmunity was first demonstrated by the observation that PD-1 knockout mice develop late-onset progressive arthritis, lupus-like glomerulonephritis and autoimmune cardiomyopathy (Nishimura et al., Immunity 11:141-151, 1999; Nishimura et al., Science 291: 319-322, 2001). Furthermore, the introduction of PD-1 deficiency in non-obese diabetic (NOD) mice accelerated significantly the incidence of diabetes, resulting in all the mice developing diabetes by 10 weeks of age (Wang et al., PNAS 102:11823-11828, 2005). In humans, PD-1 also appears to show comparable modulatory functions. Single nucleotide polymorphisms within the PD-1 gene have been linked with various autoimmune diseases, including lupus erythematosus, multiple sclerosis, Type I diabetes, rheumatoid arthritis and Grave's disease (Prokunina et al., Arthritis Rheum 50:1770, 2004; Neilson et al., Tissue Antigens 62:492, 2003; Kroner et al., Ann Neurol 58:50, 2005; Okazaki et al., Int Immunol 19:813-824, 2007); and perturbations of the PD-1 pathway have also been reported in other autoimmune diseases (Kobayashi et al., J Rheumatol 32:215, 2005; Mataki et al., Am J Gastroenterol 102:302, 2007). Finally, blockade of the PD-1 pathway by antagonistic antibodies has been associated with autoimmune side effects in cancer patients (Michot et al., Eur J Cancer 54:139-148, 2016). Therapeutic strategies that lead to activation of the PD-1 pathway provide a promising approach for the treatment of autoimmune conditions. For example, artificial dendritic cells that over-express PD-L1 have been shown to reduce spinal cord inflammation and clinical severity of experimental autoimmune encephalomyelitis in a mouse model (Hirata et al., J Immunol 174:1888-1897, 2005). Furthermore, a recombinant adenovirus expressing PD-L1, concomitant with blockade of co-stimulation molecules, has been shown to prevent lupus nephritis in BXSB mice (Ding et al., Clin Immunol 118:258-267, 2006). A number of PD-1 agonist antibodies have been developed for treatment of various autoimmune diseases in humans, (for example see, WO2013022091, WO2004056875, WO2010029435, WO2011110621, WO2015112800). However, despite the development of such reagents, there has been little evidence to suggest that soluble agents are efficient in triggering PD-1 signalling and to our knowledge only one such molecule has entered clinical testing, for the treatment of psoriasis (see NCT03337022). Administration of PD-1 agonists also has the potential to trigger systemic immune effects away from the site of disease leading to clinical toxicities. Therefore, there is a need for safer and more effective PD-1 agonist therapies for the treatment of autoimmune disease.

The inventors have surprisingly found that molecules comprising a PD-1 agonist fused to a peptide-MHC binding moiety result in efficient inhibition of PD-1 signalling.

Without being bound by theory, the inventors hypothesise that efficient inhibition of T cell activation requires localisation of a PD-1 agonist to the immune synapse. Attaching a PD-1 agonist to a moiety that binds to a disease-specific peptide-MHC, such as a TCR or TCR-like antibody, directs the agonist to the immune synapse, providing a safer and more potent strategy to modulate the PD-1 pathway.

T cell receptors (TCRs) are naturally expressed by CD4⁺ and CD8⁺ T cells. TCRs are designed to recognize short peptide antigens that are displayed on the surface of antigen presenting cells in complex with Major Histocompatibility Complex (MHC) molecules (in humans, MHC molecules are also known as Human Leukocyte Antigens, or HLA) (Davis, et al., (1998), Annu Rev Immunol 16: 523-544.). CD8⁺ T cells, which are also termed cytotoxic T cells, specifically recognize peptides bound to MHC class I and are generally responsible for finding and mediating the destruction of infected or cancerous cells.

It is desirable that TCRs for immunotherapeutic use are able to strongly recognise the target antigen, by which it is meant that the TCR should possess a high affinity and/or long binding half-life for the target antigen in order to exert a potent response. TCRs as they exist in nature typically have low affinity for target antigen (low micromolar range), thus it is often necessary to identify mutations, including but not limited to substitutions, insertions and/or deletions, that can be made to a given TCR sequence in order to improve antigen binding. For use as soluble targeting agents TCR antigen binding affinities in the nanomolar to picomolar range and with binding half-lives of several hours are preferable. It is also desirable that therapeutic TCRs demonstrate a high level of specificity for the target antigen to mitigate the risk of toxicity in clinical applications resulting from off-target binding. Such high specificity may be especially challenging to obtain given the natural degeneracy of TCR antigen recognition (Wooldridge, et al., (2012), J Biol Chem 287(2): 1168-1177; Wilson, et al., (2004), Mol Immunol 40(14-15): 1047-1055). Finally, it is desirable that therapeutic TCRs are able to be expressed and purified in a highly stable form.

### SUMMARY OF THE INVENTION

The present invention provides, as a first aspect, a bifunctional binding polypeptide comprising a pMHC binding moiety and a PD-1 agonist. The pMHC binding moiety may comprise TCR variable domains and/or antibody variable domains. The pMHC binding moiety may be a T cell receptor (TCR) or a TCR-like antibody. The pMHC binding moiety may be a heterodimeric alpha/beta TCR polypeptide pair or a single chain alpha/beta TCR polypeptide. The PD-1 agonist may be the soluble extracellular form of PD-L1 or a functional fragment thereof, the PD-L1 may comprise or consist of the sequence: FTVTVPKDLYVVEYGSNMTIECKFPVEKQLDLAALIVYWEMEDKNIIQFVHGEEDLKVQHS SYRQRARLLKDQLSLGNAALQITDVKLQDAGVYRCMISYGGADYKRITVKVNAPY. The PD-1 agonist may a full-length antibody or fragment thereof, such as a scFv antibody.

The PD-1 agonist may be fused to the C or N terminus of the pMHC binding moiety and may be fused to the pMHC binding moiety via a linker. The linker may be up to 25 amino acids in length. Preferably the linker is 2, 3, 4, 5, 6, 7 or 8 amino acids in length.

When the pMHC binding moiety is a TCR, the TCR may comprise a non-native di-sulphide bond between the constant region of the alpha chain and the constant region of the beta chain and may bind specifically to a peptide antigen.

A further aspect of the invention provides the bifunctional binding polypeptide in accordance with the first aspect of the invention for use in treating autoimmune disease, such as Alopecia Areata, Ankylosing spondylitis, Atopic dermatitis, Grave's disease, Multiple sclerosis, Psoriasis, Rheumatoid arthritis, Systemic lupus erythematosus, Type 1 diabetes and Vitiligo and Inflammatory Bowel Disease.

The invention also provides a pharmaceutical composition comprising the bifunctional binding polypeptide according to the first aspect.

A nucleic acid encoding the bifunctional binding polypeptide according to the first aspect is provided, as well as an expression vector comprising such a nucleic acid.

Further provided is a host cell comprising such a nucleic acid or such a vector, wherein the nucleic acid encoding the bifunctional binding polypeptide may be present as a single open reading frame or two distinct open reading frames encoding the alpha chain and beta chain of a TCR, respectively.

A method of making the bifunctional binding polypeptide according to the first aspect is also provided, wherein the method comprises maintaining the host cell of the invention under optional conditions for expression of the nucleic acid and isolating the bifunctional binding peptide of the first aspect.

A method of treating an autoimmune disorder comprising administering the bifunctional binding polypeptide according to the first aspect to a patient in need thereof, is also included in the invention.

### DETAILED DESCRIPTION OF THE INVENTION

The present invention provides, as a first aspect, a bifunctional binding polypeptide comprising a pMHC binding moiety and a PD-1 agonist. The pMHC binding moiety may comprise TCR variable domains. Alternatively, the pMHC binding moiety may comprise antibody variable domains. The pMHC binding moiety may be a T cell receptor (TCR) or a TCR-like antibody.

TCR sequences are most usually described with reference to IMGT nomenclature which is widely known and accessible to those working in the TCR field. For example, see: LeFranc and LeFranc, (2001). “T cell Receptor Factsbook”, Academic Press; Lefranc, (2011), Cold Spring Harb Protoc 2011(6): 595-603; Lefranc, (2001), Curr Protoc Immunol Appendix 1: Appendix 10; and Lefranc, (2003), Leukemia 17(1): 260-266. Briefly, a43 TCRs consist of two disulphide linked chains. Each chain (alpha and beta) is generally regarded as having two domains, namely a variable and a constant domain. A short joining region connects the variable and constant domains and is typically considered part of the alpha variable region. Additionally, the beta chain usually contains a short diversity region next to the joining region, which is also typically considered part of the beta variable region.

The variable domain of each chain is located N-terminally and comprises three Complementarity Determining Regions (CDRs) embedded in a framework sequence (FR). The CDRs comprise the recognition site for peptide-MHC binding. There are several genes coding for alpha chain variable (Vα) regions and several genes coding for beta chain variable (Vβ) regions, which are distinguished by their framework, CDR1 and CDR2 sequences, and by a partly defined CDR3 sequence. The Vα and Vβ genes are referred to in IMGT nomenclature by the prefix TRAV and TRBV respectively (Folch and Lefranc, (2000), Exp Clin Immunogenet 17(1): 42-54; Scaviner and Lefranc, (2000), Exp Clin Immunogenet 17(2): 83-96; LeFranc and LeFranc, (2001), “T cell Receptor Factsbook”, Academic Press). Likewise there are several joining or J genes, termed TRAJ or TRBJ, for the alpha and beta chain respectively, and for the beta chain, a diversity or D gene termed TRBD (Folch and Lefranc, (2000), Exp Clin Immunogenet 17(2): 107-114; Scaviner and Lefranc, (2000), Exp Clin Immunogenet 17(2): 97-106; LeFranc and LeFranc, (2001), “T cell Receptor Factsbook”, Academic Press). The huge diversity of T cell receptor chains results from combinatorial rearrangements between the various V, J and D genes, which include allelic variants, and junctional diversity (Arstila, et al., (1999), Science 286(5441): 958-961; Robins et al., (2009), Blood 114(19): 4099-4107.) The constant, or C, regions of TCR alpha and beta chains are referred to as TRAC and TRBC respectively (Lefranc, (2001), Curr Protoc Immunol Appendix 1: Appendix 10).

When the pMHC binding moiety is a TCR, the TCR may be non-naturally occurring and/or purified and/or engineered. More than one mutation may be present in the alpha chain variable domain and/or the beta chain variable domain relative to the native TCR. Mutations are preferably made within the CDR regions. Such mutation(s) are typically introduced in order to improve the binding affinity of the binding moiety (e.g. TCR) to the specific peptide antigen HLA complex.

The pMHC binding moiety may be a TCR-like antibody. A TCR-like antibody is the term used in the art for antibody molecules endowed with a TCR-like specificity toward peptide antigens presented by MHC, and usually have a higher affinity for antigen than native TCRs. (Dahan et al., Expert Rev Mol Med 14:e6, 2012). Such antibodies may comprise a heavy chain and a light chain, each comprising a variable region and a constant region. Functional fragments of such antibodies are encompassed by the invention, such as scFvs, Fab fragments and so on, as well known in the art.

The bifunctional binding polypeptides of the invention have the property of binding a specific peptide antigen-MHC complex. Specificity in the context of polypeptides of the invention relates to their ability to recognise target cells that present the peptide antigen-MHC complex, whilst having minimal ability to recognise target cells that do not present the peptide antigen-MHC complex.

The bifunctional binding polypeptides of the invention may have an ideal safety profile for use as therapeutic reagents. An ideal safety profile means that in addition to demonstrating good specificity, the polypeptides of the invention may have passed further preclinical safety tests. Examples of such tests include alloreactivity tests to confirm low potential for recognition of alternative HLA types.

The bifunctional binding polypeptides of the invention may be amenable to high yield purification. Yield may be determined based on the amount of material retained during the purification process (i.e. the amount of correctly folded material obtained at the end of the purification process relative to the amount of solubilised material obtained prior to refolding), and or yield may be based on the amount of correctly folded material obtained at the end of the purification process, relative to the original culture volume. High yield means greater than 1%, or more preferably greater than 5%, or higher yield. High yield means greater than 1 mg/ml, or more preferably greater than 3 mg/ml, or greater than 5 mg/ml, or higher yield.

The bifunctional binding polypeptides of the invention will have a suitable binding affinity for a peptide antigen and for PD-1. Methods to determine binding affinity (inversely proportional to the equilibrium constant K_{D}) and binding half-life (expressed as T½) are known to those skilled in the art. In a preferred embodiment, binding affinity and binding half-life are determined using Surface Plasmon Resonance (SPR) or Bio-Layer Interferometry (BLI), for example using a BIAcore instrument or Octet instrument, respectively. It will be appreciated that doubling the affinity of a binding polypeptide results in halving the K_{D}. T½ is calculated as ln 2 divided by the off-rate (k_{off}). Therefore, doubling of T½ results in a halving in k_{off}. K_{D} and k_{off} values are usually measured for soluble forms of polypeptides. To account for variation between independent measurements, and particularly for interactions with dissociation times in excess of 20 hours, the binding affinity and or binding half-life of a given polypeptide may be measured several times, for example 3 or more times, using the same assay protocol, and an average of the results taken. To compare binding data between two samples (i.e. two different polypeptides and or two preparations of the same polypeptide) it is preferable that measurements are made using the same assay conditions (e.g. temperature).

For bifunctional binding polypeptides of the invention where the pMHC binding moiety comprises TCR variable domains, the domains may be α and β variable domains. Where the pMHC binding moiety is a TCR, such TCRs may be αβ heterodimers. In certain cases, the pMHC binding moiety comprises γ and δ TCR variable domains. Where the pMHC binding moiety is a TCR, such TCRs may be γδ heterodimers.

pMHC binding moieties of the invention may comprise an extracellular alpha chain TRAC constant domain sequence and/or na extracellular beta chain TRBC1 or TRBC2 constant domain sequence. The constant domains may be truncated such that the transmembrane and cytoplasmic domains are absent. One or both of the constant domains may contain mutations, substitutions or deletions relative to the native TRAC and/or TRBC1/2 sequences. The term TRAC and TRBC1/2 also encompasses natural polymorphic variants, for example N to K at position 4 of TRAC (Bragado et al International immunology. 1994 February; 6(2):223-30).

Alternatively, rather than full-length or truncated constant domains there may be no TCR constant domains. Accordingly, the pMHC binding moiety of the invention may be comprised of the variable domains of the TCR alpha and beta chains.

When the pMHC binding moiety comprises TCR variable domains, such TCR variable domains may be in single chain format, such as for example a single chain TCR. Single chain formats include, but are not limited to, αβ TCR polypeptides of the Vα-L-Vβ, Vβ-L-Vα, Vα-Ca-L-Vβ, Vα-L-Vβ-Cβ, or Vα-Ca-L-Vβ-Cβ types, wherein Vα and Vβ are TCR α and β variable regions respectively, Cα and Cβ are TCR α and β extracellular constant regions respectively, and L is a linker sequence (Weidanz et al., (1998) J Immunol Methods. Dec 1; 221(1-2):59-76; Epel et al., (2002), Cancer Immunol Immunother. November; 51(10):565-73; WO 2004/033685; WO9918129). Where present, one or both of the extracellular constant domains may be full length, or they may be truncated and/or contain mutations as described above. In certain embodiments single chain TCR variable domains and/or single chain TCRs of the invention may have an introduced disulphide bond between residues of the respective constant domains, as described in WO 2004/033685. Single chain TCRs are further described in WO2004/033685; WO98/39482; WO01/62908; Weidanz et al. (1998) J Immunol Methods 221(1-2): 59-76; Hoo et al. (1992) Proc Natl Acad Sci USA 89(10): 4759-4763; Schodin (1996) Mol Immunol 33(9): 819-829).

For bifunctional binding polypeptides of the invention where the pMHC binding moiety is a TCR, the alpha and beta chain constant domain sequences of such a TCR may be modified by truncation or substitution to delete the native disulphide bond between Cys4 of exon 2 of TRAC and Cys2 of exon 2 of TRBC1 or TRBC2. The alpha and/or beta chain constant domain sequence(s) may have an introduced disulphide bond between residues of the respective constant domains, as described, for example, in WO 03/020763. In a preferred embodiment the alpha and beta constant domains may be modified by substitution of cysteine residues at position Thr 48 of TRAC and position Ser 57 of TRBC1 or TRBC2, the said cysteines forming a disulphide bond between the alpha and beta constant domains of the TCR. TRBC1 or TRBC2 may additionally include a cysteine to alanine mutation at position 75 of the constant domain and an asparagine to aspartic acid mutation at position 89 of the constant domain. One or both of the extracellular constant domains present in an αβ heterodimer of the invention may be truncated at the C terminus or C termini, for example by up to 15, or up to 10, or up to 8 or fewer amino acids. One or both of the extracellular constant domains present in an 43 heterodimer of the invention may be truncated at the C terminus or C termini by, for example, up to 15, or up to 10 or up to 8 amino acids. The C terminus of the alpha chain extracellular constant domain may be truncated by 8 amino acids.

A non-native disulphide bond may be present between the extracellular constant domains. Said non-native disulphide bonds are further described in WO03020763 and WO06000830. The non-native disulphide bond may be between position Thr 48 of TRAC and position Ser 57 of TRBC1 or TRBC2. One or both of the constant domains may contain one or more mutations substitutions or deletions relative to the native TRAC and/or TRBC1/2 sequences.

In another preferred format of the bifunctional binding polypeptides where the pMHC binding moiety comprises TCR variable domains, the TCR variable domains and PD-1 agonist domain(s) may be alternated on separate polypeptide chains, leading to dimerization. Such formats are described in WO2019012138. In brief, the first polypeptide chain could include (from N to C terminus) a first antibody variable domain followed by a TCR variable domain, optionally followed by a Fc domain. The second chain could include (from N to C terminus) a TCR variable domain followed by a second antibody variable domain, optionally followed by a Fc domain. Given linkers of an appropriate length, the chains would dimerise into a multi-specific molecule, optionally including a Fc domain. Molecules in which domains are located on different chains in this way may also be referred to as diabodies, which are also contemplated herein. Additional chains and domains may be added to form, for example, triabodies.

Accordingly, there is also provided herein a dual specificity polypeptide molecule selected from the group of molecules comprising a first polypeptide chain and a second polypeptide chain, wherein:
the first polypeptide chain comprises a first binding region of a variable domain (VD1) of a PD-1 agonist antibody, and a first binding region of a variable domain (VR1) of a TCR specifically binding to an MHC-associated peptide epitope, and a first linker (LINK1) connecting said domains;
the second polypeptide chain comprises a second binding region of a variable domain (VR2) of a TCR specifically binding to an MHC-associated peptide epitope, and a second binding region of a variable domain (VD2) of a PD-1 agonist antibody, and a second linker (LINK2) connecting said domains;
wherein said first binding region (VD1) and said second binding region (VD2) associate to form a first binding site (VD1)(VD2);
said first binding region (VR1) and said second binding region (VR2) associate to form a second binding site (VR1)(VR2) that binds said MHC-associated peptide epitope;
wherein said two polypeptide chains are fused to human IgG hinge domains and/or human IgG Fc domains or dimerizing portions thereof; and
wherein the said two polypeptide chains are connected by covalent and/or non-covalent bonds between said hinge domains and/or Fc-domains; and
wherein said dual specificity polypeptide molecule is capable of simultaneously agonising PD-1 and binding the MHC-associated peptide epitope, and dual specificity polypeptide molecules, wherein the order of the binding regions in the two polypeptide chains is selected from VD1-VR1 and VR2-VD2 or VD1-VR2 and VR1-VD2, or VD2-VR1 and VR2-VD1 or VD2-VR2 and VR1-VD1 and wherein the domains are either connected by LINK1 or LINK2.

The PD-1 agonist may correspond to the soluble extracellular region of PD-L1 (Uniprot ref: Q9NZQ7) or PD-L2 (Q9BQ51) or a functional fragment thereof. The PD-L1 may comprise or consist of a sequence as set out below.

Full length PD-L1 has the sequence set out below:

FTVTVPKDLYVVEYGSNMTIECKFPVEKQLD
LAALIVYWEMEDKNIIQFVHGEEDLKVQHSS
YRQRARLLKDQLSLGNAALQITDVKLQDAGV
YRCMISYGGADYKRITVKVNAPYNKINQRIL
VVDPVTSEHELTCQAEGYPKAEVIWTSSDHQ
VLSGKTTTTNSKREEKLFNVTSTLRINTTTN
EIFYCTFRRLDPEENHTAELVIPELPLAHPP
NER

A truncated form of PD-L1 may be fused to the pMHC binding moiety, provided it retains the ability to bind and agonise PD-1. Such a truncated fragment may be as set out in the sequence below:

FTVTVPKDLYVVEYGSNMTIECKFPVEKQLD
LAALIVYWEMEDKNIIQFVHGEEDLKVQHSS
YRQRARLLKDQLSLGNAALQITDVKLQDAGV
YRCMISYGGADYKRITVKVNAPY

Alternatively, shorter or longer truncations may also be fused to the pMHC binding moiety.

The PD-1 agonist may a full-length antibody or fragment thereof, such as a scFv antibody or a Fab fragment, or a nanobody. Examples of such antibodies are provided in WO2011110621 and WO2010029434 and WO2018024237. The antibody molecules of the present invention may comprise a complete antibody molecule having full length heavy and light chains or a fragment thereof and may be, but are not limited to Fab, modified Fab, Fab′, modified Fab′, F(ab′)2, Fv, single domain antibodies (e.g. VH or VL or VHH), scFv, bi, tri or tetra-valent antibodies, Bis-scFv, diabodies, triabodies, tetrabodies nanobodies and epitope-binding fragments of any of the above.

The PD-1 agonist may be fused to the C or N terminus of the pMHC binding moiety and may be fused to the pMHC binding moiety via a linker which may be 2, 3, 4, 5, 6, 7 or 8 amino acids in length. Linkers may be 10, 12, 15, 16, 18, 20 or 25 amino acids in length. The linker sequence may be repeated to form a longer linker. Each linker may be formed on one, two three or four repeats of a shorter linker sequence. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. The linker may be up to 25 amino acids in length. Often the linker sequence will be less than about 12, such as less than 10, or from 2-8 amino acids in length. Examples of suitable linkers that may be used in TCRs of the invention include but are not limited to: GGGGS, GGGSG, GGSGG, GSGGG, GSGGGP, GGEPS, GGEGGGP, and GGEGGGSEGGGS (as described in WO2010/133828).

The bifunctional binding polypeptide of the present invention may further comprise a pK modifying moiety. Where an immunoglobulin Fc domain is used, it may be any antibody Fc region. The Fc region is the tail region of an antibody that interacts with cell surface Fc receptors and some proteins of the complement system. The Fc region typically comprises two polypeptide chains both having two or three heavy chain constant domains (termed CH2, CH3 and CH4), and a hinge region. The two chains being linked by disulphide bonds within the hinge region. Fc domains from immunoglobulin subclasses IgG1, IgG2 and IgG4 bind to and undergo FcRn mediated recycling, affording a long circulatory half-life (3-4 weeks). The interaction of IgG with FcRn has been localized in the Fc region covering parts of the CH2 and CH3 domain. Preferred immunoglobulin Fc for use in the present invention include, but are not limited to Fc domains from IgG1 or IgG4. Preferably the Fc domain is derived from human sequences. The Fc region may also preferably include KiH mutations which facilitate dimerization, as well as and mutations to prevent interaction with activating receptors i.e. functionally silent molecules. The immunoglobulin Fc domain may be fused to the C or N terminus of the other domains (i.e., the TCR variable domains or immune effector). The immunoglobulin Fc may be fused to the other domains (i.e., the TCR variable domains or immune effector) via a linker. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length, The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used multi-domain binding molecules of the invention include, but are not limited to: GGGSGGGG, GGGGS, GGGSG, GGSGG, GSGGG, GSGGGP, GGEPS, GGEGGGP, and GGEGGGSEGGGS (as described in WO2010/133828). Where the immunoglobulin Fc is fused to the TCR, it may be fused to either the alpha or beta chains, with or without a linker. Furthermore, individual chains of the Fc may be fused to individual chains of the TCR.

Preferably the Fc region may be derived from the IgG1 or IgG4 subclass. The two chains may comprise CH2 and CH3 constant domains and all or part of a hinge region. The hinge region may correspond substantially or partially to a hinge region from IgG1, IgG2, IgG3 or IgG4. The hinge may comprise all or part of a core hinge domain and all or part of a lower hinge region. Preferably, the hinge region contains at least one disulphide bond linking the two chains.

The Fc region may comprise mutations relative to a WT sequence. Mutations include substitutions, insertions and deletions. Such mutations may be made for the purpose of introducing desirable therapeutic properties. For example, to facilitate heterodimersation, knobs into holes (KiH) mutations maybe engineered into the CH3 domain. In this case, one chain is engineered to contain a bulky protruding residue (i.e. the knob), such as Y, and the other is chain engineered to contain a complementary pocket (i.e. the hole). Suitable positions for KiH mutations are known in the art. Additionally or alternatively mutations may be introduced that abrogate or reduce binding to Fcy receptors and or increase binding to FcRn, and/or prevent Fab arm exchange, or remove protease sites.

The PK modifying moiety may also be an albumin-biding domain, which may also act to extend half-life. As is known in the art, albumin has a long circulatory half-life of 19 days, due in part to its size, being above the renal threshold, and by its specific interaction and recycling via FcRn. Attachment to albumin is a well-known strategy to improve the circulatory half-life of a therapeutic molecule in vivo. Albumin may be attached non-covalently, through the use of a specific albumin binding domain, or covalently, by conjugation or direct genetic fusion. Examples of therapeutic molecules that have exploited attachment to albumin for improved half-life are given in Sleep et al., Biochim Biophys Acta. 2013 December; 1830(12):5526-34.

The albumin-binding domain may be any moiety capable of binding to albumin, including any known albumin-binding moiety. Albumin binding domains may be selected from endogenous or exogenous ligands, small organic molecules, fatty acids, peptides and proteins that specifically bind albumin. Examples of preferred albumin binding domains include short peptides, such as described in Dennis et al., J Biol Chem. 2002 Sep. 20; 277(38):35035-43 (for example the peptide QRLMEDICLPRWGCLWEDDF); proteins engineered to bind albumin such as antibodies, antibody fragments and antibody like scaffolds, for example Albudab® (O'Connor-Semmes et al., Clin Pharmacol Ther. 2014 December; 96(6):704-12), commercially provided by GSK and Nanobody® (Van Roy et al., Arthritis Res Ther. 2015 May 20; 17:135), commercially provided by Ablynx; and proteins based on albumin binding domains found in nature such as Streptococcal protein G Protein (Stork et al., Eng Des Sel. 2007 November; 20(11):569-76), for example Albumod® commercially provided by Affibody

Preferably, albumin is human serum albumin (HSA). The affinity of the albumin binding domain for human albumin may be in the range of picomolar to micromolar. Given the extremely high concentration of albumin in human serum (35-50 mg/ml, approximately 0.6 mM), it is calculated that substantially all of the albumin binding domains will be bound to albumin in vivo.

The albumin-binding moiety may be linked to the C or N terminus of the other domains (i.e., the TCR variable domains or immune effector). The albumin-binding moiety may be linked to the other domains (i.e., the TCR variable domains or immune effector) via a linker. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. The liker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used in multi-domain binding molecules of the invention include, but are not limited to: GGGSGGGG, GGGGS, GGGSG, GGSGG, GSGGG, GSGGGP, GGEPS, GGEGGGP, and GGEGGGSEGGGS (as described in WO2010/133828). Where the albumin-binding moiety is linked to the TCR, it may be linked to either the alpha or beta chains, with or without a linker.

A further aspect of the invention provides the bifunctional binding polypeptide in accordance with the first aspect of the invention for use in treating autoimmune disease, such as Alopecia Areata, Ankylosing spondylitis, Atopic dermatitis, Grave's disease, Multiple sclerosis, Psoriasis, Rheumatoid arthritis, Systemic lupus erythematosus, Type 1 diabetes, Vitiligo, Inflammatory Bowel Disease, Crohn's disease, ulcerative colitis, coeliac disease, eye diseases (e.g. uveitis), cutaneous lupus and lupus nephritis, and autoimmune disease in cancer patients caused by PD-1/PD-L1 antagonists.

The invention also provides the bifunctional binding polypeptide in accordance with the first aspect of the invention for use in the treatment or prophylaxis of pain, particularly pain associated with inflammation.

Optionally, the bifunctional polypeptide of the invention is for use in the treatment of type 1 diabetes, inflammatory bowel disease and rheumatoid arthritis.

The invention also provides a pharmaceutical composition comprising the bifunctional binding polypeptide according to the first aspect.

In a further aspect, the present invention provides nucleic acid encoding a bifunctional binding polypeptide of the invention. In some embodiments, the nucleic acid is cDNA. In some embodiments the nucleic acid may be mRNA. In some embodiments, the invention provides nucleic acid comprising a sequence encoding an a chain variable domain of a TCR of the invention. In some embodiments, the invention provides nucleic acid comprising a sequence encoding a β chain variable domain of a TCR of the invention. In some embodiments, the invention provides nucleic acid comprising a sequence encoding a light chain of a TCR-like antibody. In some embodiments, the invention provides nucleic acid comprising a sequence encoding a heavy chain of a TCR-like antibody. In some embodiments, the invention provides nucleic acid comprising a sequence encoding all or part of a PD-1 agonist, for example PD-L1 or a truncated from thereof, or all or part of a agonistic PD-1 antibody, such as the light chain and/or heavy chain of such an antibody. The nucleic acid may be non-naturally occurring and/or purified and/or engineered. The nucleic acid sequence may be codon optimised, in accordance with expression system utilised. As is known to those skilled in the art, expression systems may include bacterial cells such as E. coli, or yeast cells, or mammalian cells, or insect cells, or they may be cell free expression systems.

In another aspect, the invention provides a vector which comprises a nucleic acid of the invention. Preferably the vector is a suitable expression vector.

The invention also provides a cell harbouring a vector of the invention. Suitable cells include, bacterial cells such as E. coli, or yeast cells, or mammalian cells, or insect cells. The vector may comprise nucleic acid of the invention encoding in a single open reading frame, or two distinct open reading frames, encoding the alpha chain and the beta chain of a TCR respectively, or a light chain or heavy chain of a TCR-like antibody, respectively.

Another aspect provides a cell harbouring a first expression vector which comprises nucleic acid encoding the alpha chain/light chain of a TCR/TCR-like antibody of the polypeptide of the invention, and a second expression vector which comprises nucleic acid encoding the beta chain/heavy chain of a TCR/TCR-like antibody of the invention. The cells of the invention may be isolated and/or recombinant and/or non-naturally occurring and/or engineered.

As is well-known in the art, polypeptides may be subject to post translational modifications. Glycosylation is one such modification, which comprises the covalent attachment of oligosaccharide moieties to defined amino acids in the TCR/TCR-like antibody/PD-L1 or

PD-1 antibody or other PD-1 agonist. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis et al., (2009) Nat Rev Drug Discov March; 8(3):226-34.). For soluble TCRs of the invention glycosylation may be controlled, by using particular cell lines for example (including but not limited to mammalian cell lines such as Chinese hamster ovary (CHO) cells or human embryonic kidney (HEK) cells), or by chemical modification. Such modifications may be desirable, since glycosylation can improve pharmacokinetics, reduce immunogenicity and more closely mimic a native human protein (Sinclair and Elliott, (2005) Pharm Sci. August; 94(8):1626-35).

For administration to patients, the bifunctional binding polypeptides of the invention, may be provided as part of a sterile pharmaceutical composition together with one or more pharmaceutically acceptable carriers or excipients. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, such as parenteral (including subcutaneous, intramuscular, intrathecal or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. a suitable dose range for a bifunctional binding polypeptide may be in the range of 25 ng/kg to 50 μg/kg or 1 μg to 1 g. A physician will ultimately determine appropriate dosages to be used.

Bifunctional binding polypeptides, pharmaceutical compositions, vectors, nucleic acids and cells of the invention may be provided in substantially pure form, for example, at least 80%, at least 85%, at least 90%, at least 91%, at least 92%, at least 93%, at least 94%, at least 95%, at least 96%, at least 97%, at least 98%, at least 99% or 100% pure.

Further provided is a host cell comprising such a nucleic acid or such a vector, wherein the nucleic acid encoding the bifunctional binding polypeptide may be present as a single open reading frame or two distinct open reading frames encoding the alpha chain and beta chain of a TCR, respectively.

A method of making the bifunctional binding polypeptide according to the first aspect is also provided, wherein the method comprises maintaining the host cell of the invention under optional conditions for expression of a nucleic acid of the invention and isolating the bifunctional binding peptide of the first aspect.

Preferred features of each aspect of the invention are as for each of the other aspects mutatis mutandis. The prior art documents mentioned herein are incorporated to the fullest extent permitted by law.

The invention is now described with reference to the following non-limiting examples and figures in which:

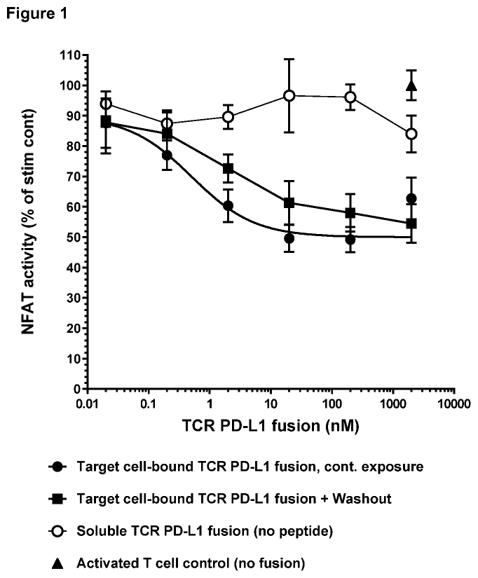

FIG. 1 shows dose-dependent inhibition of NFAT reporter activity with a bifunctional polypeptide of the invention comprising a soluble TCR and a truncated form of PD-L1, in the presence of peptide-pulsed target cells.

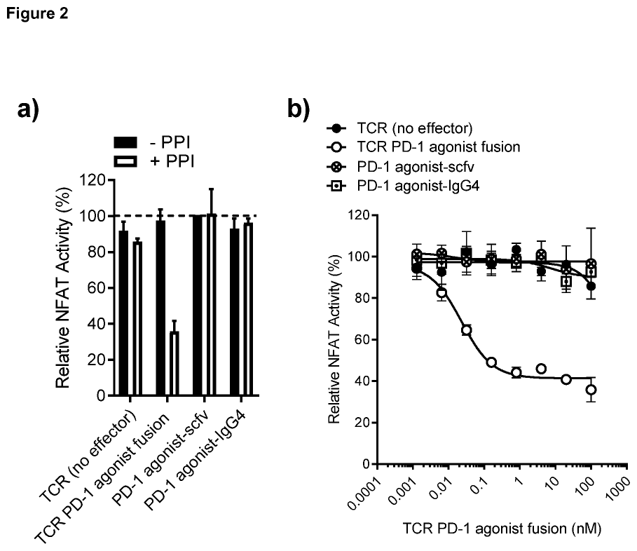

FIG. 2 shows inhibition of NFAT reporter activity with a bifunctional polypeptide of the invention comprising a soluble TCR and a PD-1 agonist scFv antibody fragment, in the presence of peptide-pulsed target cells.

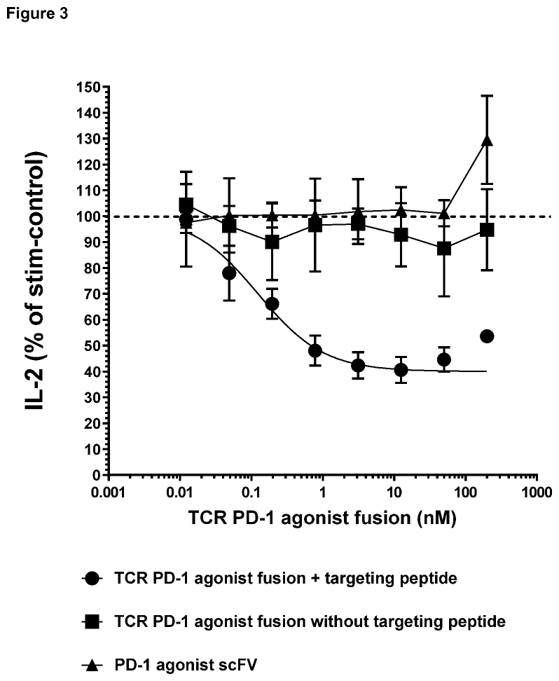

FIG. 3 shows inhibition of primary human t cells activation with a bifunctional polypeptide of the invention comprising a soluble TCR and a PD-1 agonist scFv antibody fragment, in the presence of peptide-pulsed target cells.

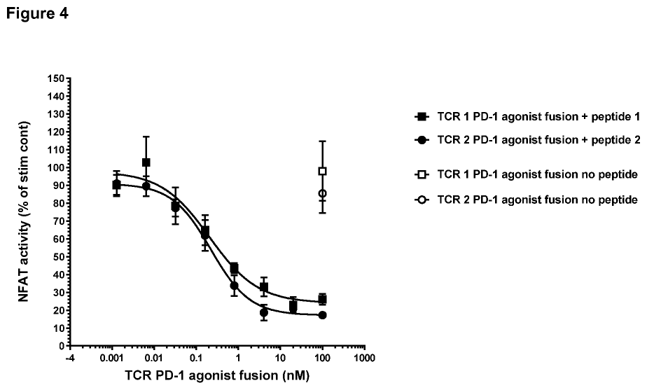

FIG. 4 shows inhibition of NFAT reporter activity with a bifunctional polypeptide of the invention comprising one of two soluble TCRs with differing specificity, and a PD-1 agonist scFv antibody fragment, in the presence of peptide-pulsed target cells.

### EXAMPLES

### Example 1

The following example demonstrates that a PD-1 agonist fused to a soluble TCR can effectively inhibit T cell activation when targeted to the immune synapse.

The soluble TCR used in this bifunctional binding polypeptide is an affinity-enhanced version of a native TCR that specifically recognises a HLA-A*02 restricted peptide derived from human pre-pro insulin (such molecules are described in WO2015092362). The PD-1 agonist is a truncated version of the extracellular region of PD-L1 comprising the PD-1 interaction site (Zak et al., Structure 23:2341-2348, 2015). PD-L1 is fused to the N-terminus of the TCR alpha chain via a standard 5 amino acid linker.

A Jurkat NFAT luciferase PD-1 reporter assay was used for measuring TCR-PD1 agonist fusion molecule-mediated inhibition of T cell NFAT activity in the presence of HEK293T antigen presenting target cells.

### Methods

### Expression, Refolding and Purification of TCR-PD1 Agonist Fusion Molecules

Expression of TCR-PD1 agonist fusion molecules was performed using the high-yield transient expression system based on suspension-adapted Chinese Hamster Ovary (CHO) cells (ExpiCHO Expression system, Thermo Fisher). Cells were co-transfected according to the manufacturer's instructions, using mammalian expression plasmids containing the TCR chains fused to a PD-1 agonist. Following the harvest, clarification of cell culture supernatants was done by centrifuging the supernatant at 4000-5000×g for 30 minutes in a refrigerated centrifuge. Supernatants were filtered through a 0.22-μm filter and collected for further purification.

Alternatively, the expression of TCR-PD1 agonist fusion molecules was carried out using E. coli as the host organism. Expression plasmids containing alpha and beta chain were separately transformed into BL21pLysS E. coli strain and plated onto LB-agar plate containing 100 μg/mL ampicillin. Loopful colonies from each transformation were picked and grown in LB media (with 100 μg/mL ampicillin and 1% glucose) at 37° C. until OD600 reached ˜0.5-1.0. The LB starter culture was then added to autoinduction media (Foremedium) and cells grown for 37° C.˜3 hours followed by 30° C. overnight. Cells were harvested by centrifugation and lysed in Bugbuster (Novagen). Inclusion bodies (IBs) were extracted by performing two Triton wash (50 mM Tris pH 8.1, 100 mM, NaCl, 10 mM EDTA, 0.5% Triton) to remove cell debris and membrane. Each time IBs were harvested by centrifugation @10000 g for 5 minutes. To remove detergent, IBs were washed with 50 mM Tris pH8.1, 100 mM NaCl and 10 mM EDTA. IBs were finally re-suspended in 50 mM Tris pH8.1, 100 mM NaCl and 10 mM EDTA buffer. To measure the protein yield, IBs were solubilized in 8M Urea buffer and concentration determined by absorbance at 280 nM.

For refolding alpha and beta chains were mixed at 1:1 molar ratio and denatured for 30 minutes at 37° C. in 6 M Guanidine-HCl, 50 mM Tris pH8.1, 100 mM NaCl, 10 mM EDTA, 20 mM DTT. The denatured chains were then added to refold buffer consisting of 4 M Urea, 100 mM Tris pH 8.1, 0.4 M L-Arginine, 2 mM EDTA, 1 mM Cystamine and 10 mM Cysteamine and incubated for 10 minutes with constant stirring. The refold buffer containing the denatured chains was dialysed in Spectra/Por 1 membrane against 10× volume of H₂O for ˜16 hours, 10× volume of 10 mM Tris pH 8.1 for ˜7 hours and 10× volume of 10 mM Tris pH8.1 for ˜16 hours.

Soluble proteins obtained from either mammalian or E. coli expression systems were purified on the AKTA pure (GE healthcare) using a POROS 50 HQ (Thermo Fisher Scientific) anion exchange column using 20 mM Tris pH 8.1 as loading buffer and 20 mM Tris pH8.1 with 1M NaCl as binding and elution buffer. The protein was loaded on the column and eluted with a gradient of 0-50% of elution buffer. Fractions containing the protein were pooled and diluted 20× (volume/volume) in 20 mM MES pH6.0 for second step cation exchange chromatography on POROS 50 HS (Thermos Fisher Scientific) column using 20 mM MES pH6.0 and 20 mM MES pH6.0, 1M NaCl as binding and elution buffer respectively. Bound protein from cation exchange column was eluted using 0-100% gradient of elution buffer. Cation-exchange fractions containing the protein were pooled and further purified on Superdex 200 HR (GE healthcare) gel filtration column using PBS as running buffer. Positive fractions from gel filtration were pooled, concentrated and stored at −80° C. until required.

### Jurkat NFAT Luc-PD-1 Reporter Assay

HLA-A*02 positive HEK293T target cells were transiently transfected with a TCR activator plasmid (BPS Bioscience, Cat no: 60610) and pulsed with the relevant peptide recognised by the TCR-PD1 agonist fusion molecule. Target cells were then incubated with different concentrations of TCR-PD1 agonist fusion molecule to allow binding to cognate peptide-HLA-A2 complex. Jurkat NFAT Luc PD-1 effector cells, which constitutively express PD-1, were added to the target cells and NFAT activity determined after 18-20 h. Experiments were performed with or without washout (post-TCR-PD1 agonist fusion molecule binding). A further control was performed using non-pulsed target cells. TCR Activator/PD-L1 transfected HEK293T A2B2M target cells were included as positive controls.

### Results

The data shown in FIG. 1, demonstrates that dose-dependent inhibition of NFAT reporter activity is observed with TCR-PD1 agonist fusion molecules in the presence of peptide-pulsed target cells, with or without wash out. Crucially, minimal inhibition was observed with non-pulsed target cells indicating that targeting to the immune synapse is critical for PD-1 agonist activity.

### Example 2

The following example provides further evidence that a PD-1 agonist fused to a soluble TCR can effectively inhibit T cell activation when targeted to the immune synapse.

The experimental system and methods used in this example were the same as those described in Example 1, except that in this case the PD-1 agonist portion of the TCR-PD1 agonist fusion molecule was a scFv antibody fragment, such as described in WO2011110621.

The Jurkat NFAT luciferase PD-1 reporter assay described in Example 1 was used for measuring TCR-PD1 agonist fusion molecule-mediated inhibition of T cell NFAT activity in the presence of HEK293T antigen presenting target cells.

### Results

As shown in FIG. 2a, substantial inhibition of NFAT activity (>60%) was observed in peptide pulsed cells (labelled +PPI) treated with 100 nM TCR-PD1 agonist fusion molecule; whereas minimal inhibition was seen in non-pulsed target cells (labelled −PPI) treated with the TCR-PD1 agonist fusion molecule. Control experiments, using either the soluble TCR alone, or the PD-1 agonist alone (in both scFv or IgG4 format), showed no inhibition of reporter activity, indicating that targeting of the PD-1 agonist to the immune synapse is required for PD-1 agonist activity. FIG. 2b further shows dose-dependent inhibition of NFAT activity. Again, only the TCR-PD1 agonist fusion molecule format is able to inhibit NFAT activity. Non-targeted PD-1 agonist antibody is not able to inhibit activity.

Taken together, these results demonstrate that targeting the PD-1 agonist to the immune synapse is critical for PD-1 agonist activity.

### Example 3

The following example provides further evidence that a PD-1 agonist fused to a soluble TCR can effectively inhibit T cell activation when targeted to the immune synapse.

The TCR-PD1 agonist fusion molecule used in this example was the same as described in Example 2, in which the PD1 agonist is a scFv antibody fragment.

In this case an alternative assay was used to assess the effect of TCR-PD1 agonist fusion molecules on primary human T cell function.

### Method

### Primary Human T Cell Assay

Primary human T cells were isolated from freshly prepared PBMCs using a pan-T cell isolation kit (Miltenyi, cat no: 130-096-535). HLA-A*02 positive Raji B cells (Raji A2B2M) were pre-loaded with staphylococcal enterotoxin B (SEB, 100 ng/ml, Sigma S4881) for 1 h and then irradiated with 33Gy. For pre-activation, primary human T cells were incubated with SEB-loaded Raji A2B2M target cells at a 1:1 ratio, using 1×10E6 cells/ml of each cell type in 24-well cell culture plates. Primary human T cells were incubated for 10 days with SEB-loaded Raji A2B2M cells, with IL-2 (50 U/ml) added at d 3 and d 7. On day 10 pre-activated T cells were washed and re-suspended in fresh media. Fresh Raji A2B2M cells were pulsed with 20 μM of the relevant peptide recognised by TCR-PD1 agonist fusion molecules, or left non-pulsed for 2 h. Raji A2B2M cells were loaded with SEB (10 ng/ml) for the final 1 h of peptide pulsing and then irradiated with 33Gy. Raji A2B2M cells were plated into 96-well cell culture plates at 1×10E5 cells/well and then pre-incubated with TCR-PD1 agonist fusion molecules titrations for 1 h. Pre-activated T cells were added to the Raji A2B2M target cells at 1×10E5 cells/well and incubated for 48 h. Supernatants were collected and IL-2 levels were determined using an MSD ELISA.

### Results

The data shown in FIG. 3 demonstrate that TCR-PD1 agonist fusion molecules dose-dependently inhibits primary human T cell IL-2 production in the presence of peptide pulsed target cells, whereas non-targeted TCR-PD1 agonist fusion molecules (i.e. with non-pulsed target cells) or the PD-1 agonist scFv alone do not. These data demonstrate that targeting PD-1 agonist to the immune synapse leads to PD-1 agonist activity in primary cells

### Example 4

The following example demonstrates the same technical effect is observed using TCRs that recognise alternative antigens.

The experimental system and methods used in this example were the same as those described in Example 2. In this case a PD-1 agonist antibody was fused to two different soluble TCRs.

The Jurkat NFAT luciferase PD-1 reporter assay described in Example 1 was used for measuring TCR-PD1 agonist fusion molecule-mediated inhibition of T cell NFAT activity in the presence of HEK293T antigen presenting target cells.

### Results

As shown in FIG. 4, potent and dose dependent inhibition was observed with two TCR-PD1 agonist fusion molecules (comprising a PD-1 agonist scFv antibody fragment fused to either TCR 1 or TCR 2) administered in the presence of target cells pulsed with their respective peptides (peptides 1 or 2). For both TCR-PD1 agonist fusion molecules, minimal activity was observed when the study was conducted without the presence of targeting peptide.

These results demonstrate that TCR-PD1 agonist fusion molecules can be directed to different tissues using soluble TCRs with specificities for different pMHC and facilitate targeted inhibition of T cell activity.

1. A bifunctional binding polypeptide comprising a pMHC binding moiety and a PD-1 agonist.

2. A bifunctional binding polypeptide according to claim 1, wherein the pMHC binding moiety comprises TCR variable domains and/or antibody variable domains.

3. A bifunctional binding polypeptide according to claim 1, wherein the pMHC binding moiety is a T cell receptor (TCR) or a TCR-like antibody.

4. A bifunctional binding polypeptide according to any preceding claim, wherein the pMHC binding moiety is a heterodimeric alpha/beta TCR polypeptide pair.

5. A bifunctional binding polypeptide according to any preceding claim, wherein the pMHC binding moiety is a single chain alpha/beta TCR polypeptide.

6. A bifunctional binding polypeptide according to any one of claims 3-5, wherein the TCR comprises a non-native di-sulphide bond between the constant region of the alpha chain and the constant region of the beta chain.

7. A bifunctional binding polypeptide according to any one of claims 3-6, wherein the TCR binds specifically to a peptide antigen.

8. A bifunctional binding polypeptide according to any preceding claim, wherein the PD-1 agonist is PD-L1 or a functional fragment thereof.

9. A bifunctional binding polypeptide according to claim 8, wherein the PD-L1 comprises or consists of the sequence: FTVTVPKDLYVVEYGSNMTIECKFPVEKQLDLAALIVYWEMEDKNIIQFVHGEEDLKVQHS SYRQRARLLKDQLSLGNAALQITDVKLQDAGVYRCMISYGGADYKRITVKVNAPY

10. A bifunctional binding polypeptide according to any one of claims 1-7, wherein the PD-1 agonist is a full-length antibody or fragment thereof.

11. A bifunctional binding polypeptide according to claim 10, wherein the PD-1 agonist is a scFv antibody.

12. A bifunctional binding polypeptide according to any preceding claim, wherein the PD-1 agonist is fused to the C or N terminus of the pMHC binding moiety.

13. A bifunctional binding polypeptide according to any preceding claim, wherein the PD-1 agonist is fused to the pMHC binding moiety via a linker.

14. A bifunctional binding polypeptide according to claim 13, wherein the linker is 2, 3, 4, 5, 6, 7 or 8 amino acids in length.

15. A pharmaceutical composition comprising the bifunctional binding polypeptide according to any one of claims 1-14.

16. A nucleic acid encoding the bifunctional binding polypeptide according to any one of claims 1-14.

17. An expression vector comprising the nucleic acid of claim 16.

18. A host cell comprising the nucleic acid of claim 16 or the vector of claim 17, optionally wherein the nucleic acid encoding the bifunctional binding polypeptide is present as a single open reading frame or two distinct open reading frames encoding the alpha chain and beta chain respectively.

19. A method of making the bifunctional binding polypeptide according to any one of claims 1-14 comprising maintaining the host cell of claim 18 under optional conditions for expression of the nucleic acid and isolating the bifunctional binding peptide.

20. A bifunctional binding polypeptide according to any one of claims 1-14, a pharmaceutical composition of claim 15, a nucleic acid of claim 16 and/or a vector of claim 17, for use in medicine, particularly for treating autoimmune disease or use in the treatment or prophylaxis of pain, particularly pain associated with inflammation

21. A bifunctional binding polypeptide, pharmaceutical composition, nucleic acid and/or vector for use according to claim 20, wherein the autoimmune disease is one of Alopecia Areata, Ankylosing spondylitis, Atopic dermatitis, Grave's disease, Multiple sclerosis, Psoriasis, Rheumatoid arthritis, Systemic lupus erythematosus, Type 1 diabetes and Vitiligo, Inflammatory Bowel Disease, Crohn's disease, ulcerative colitis, coeliac disease, eye diseases (e.g. uveitis), cutaneous lupus and lupus nephritis, and autoimmune disease in cancer patients caused by PD-1/PD-L1 antagonists.

22. A method of treating an autoimmune disorder comprising administering the bifunctional binding polypeptide according to any one of claims 1-14, the pharmaceutical composition of claim 15, the nucleic acid of claim 16 and/or the vector of claim 17 to a patient in need thereof.


SEQUENCE LISTING (2 sequences)

SEQ ID NO: 1 - protein, 116 aa, Artificial Sequence
  Truncated fragment PD-L1
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPY

SEQ ID NO: 2 - protein, 220 aa, Artificial Sequence
  Full length PD-L1
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER